(19) United States
(12) Patent Application Publication
(10) Pub. No.: US 2023/0218309 A1
(43) Pub. Date: Jul. 13, 2023

(54) ADAPTER FOR AN ELECTRICAL MODULAR CATHETER SYSTEM

(71) Applicant: COVELLUS LLC, BELMAR, NJ (US)

(72) Inventor: BRADLEY BEACH, BELMAR, NJ (US)

(21) Appl. No.: 18/095,992

(22) Filed: Jan. 11, 2023

Related U.S. Application Data
(60) Provisional application No. 63/298,282, filed on Jan. 11, 2022.

(51) Int. Cl. A61B 17/22; A61M 39/10

(52) U.S. Cl. CPC A61B 17/22022; A61M 39/10; A61B 2017/22008; A61M 2039/1022; A61M 2039/1077; A61M 25/10

(57) ABSTRACT

An adapter for a medical device catheter includes a proximal portion and a distal portion, at least two electrodes positioned to create an electrode gap between the electrodes, an attachment mechanism configured to secure the adapter to a distal end of a medical device catheter, and an electrical connector at the proximal portion and in electrical communication with the electrodes.

### CROSS REFERENCE TO RELATED APPLICATIONS

This application claims the benefit of U.S. Provisional Application No. 63/298,282 filed Jan. 11, 2022, the entire contents of which are hereby incorporated by reference.

### BACKGROUND

The present disclosure relates generally to a design of an adapter for a medical device for use in the body, and more specifically to an adapter intended to convert or augment the medical device, for example a catheter, such that the purpose or configuration of the medical device is modified or expanded.

Catheter type devices are typically long tubular structures with an inner lumen suitable for a guidewire used to navigate the vasculature, inject contrast or therapeutic materials, aspirate thrombus, or provide a means to deliver other devices or therapies to a target site within the vasculature or other body lumen. Catheter type devices are typically inserted through a small opening in the skin or another opening under visual guidance and tracked to the target location within the body. Catheters for minimally invasive procedures are typically one-piece, unitary constructions combining structural, therapeutic and diagnostic elements at the distal end of the catheter.

U.S. Patent Application Publication No. 2007/0244440 discloses a medical device including a catheter with an expandable tip for use with at least two different sizes of wire guides. The catheter includes a wire guide lumen sized to receive a first wire guide of a first diameter. The catheter may also include a tip lumen that extends in a distal direction from a first opening in communication with the wire guide lumen to a second opening. The first opening is sized to receive the first wire guide, and the second opening is sized to receive a second wire guide of a smaller diameter than the first wire guide. The catheter also includes one or more longitudinal expansion features capable of radially expanding the tip lumen to receive a wire guide of a diameter up to the first diameter through the second opening.

U.S. Pat. No. 8,100,884 discloses an adapter assembly for connecting a catheter assembly to a tunneler having a generally tubular body having a first end, a second end and a longitudinal axis extending there through between the first end and the second end. The first end of the adapter is constructed to engage the proximal end of a trocar. The second end of the adapter is constructed to releasably engage at least one catheter lumen. A slider is disposed about the adapter and is longitudinally slidable along the adapter. When the slider is slid towards the second end of the adapter, the slider engages a plurality of legs on the adapter and biases the plurality of legs toward each other and the longitudinal axis of the adapter.

U.S. Pat. No. 8,523,840 discloses coupler assemblies to be used with a catheter to connect a proximal end of the catheter to extracorporeal medical equipment. An exemplary coupler assembly includes a spherical linkage coupler for a catheter. The coupler comprises a first cylinder portion for connecting to a structure, and a second cylinder portion for connecting to a distal end of a body of the catheter. The coupler also comprises a spherical linkage including at least two link arms. Each of the two link arms are connected on one end to the first cylinder portion and on the other end to the second cylinder portion. The two link arms connect a portion of the structure to the distal end of the catheter and enable the structure to move relative to the distal end of the catheter in response to an external force exerted on the structure.

U.S. Pat. Nos. 9,282,991; 9,808,276; 7,976,557; and U.S. Publication No. 2006/0259005 describe variations of a method of delivering a therapeutic agent, such as a drug, using a cutting balloon wherein the cutting or scoring members may comprise the therapeutic agent coated thereon. The cutting or scoring members are integral with the construction of the balloon and catheter system itself.

U.S. Publication No. 2008/0275427 describes a catheter connection system to connect catheter tubes together to form a secure and leak resistant connection. As described the connection system includes a threaded connector inserted into an end of a catheter lumen where an inner portion of the catheter lumen is elastically compliant to conform to the threaded structure of the connector.

U.S. Pat. No. 8,956,371 describes a shockwave balloon catheter system that uses shockwaves generated inside the inflatable balloon of an angioplasty balloon catheter to aid in treating vascular lesions blocking blood vessels. The shockwave can aid in breaking up calcium deposits in these vascular lesions. Similar shockwave technology has been used in lithotripter medical devices to help break up kidney stones in the body, as described in U.S. Pat. No. 5,047,685, for example.

It is desirable to provide an improved adapter and modular system designed with features that expand, augment, or modify the configuration or intended use of a medical device or parent module, such as by providing lithotripsy functionality. The adapter including geometry, mechanical and/or thermal properties to expeditiously attach to the medical device, such as a catheter.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, an adapter for a medical device catheter includes a proximal portion and a distal portion, at least two electrodes positioned to create an electrode gap between the electrodes, an attachment mechanism configured to secure the adapter to a distal end of a medical device catheter, and an electrical connector at the proximal portion and in electrical communication with the electrodes.

In another aspect, the electrodes are configured to generate cavitation bubbles when powered by a high voltage pulse generator.

### BRIEF DESCRIPTION OF THE DRAWINGS

FIG. 1 is a schematic, perspective, view of an adapter according to the present disclosure.

FIG. 2 is an enlarged detailed view of FIG. 1, showing part of a distal portion of the adapter, the attachment mechanism, and other features.

FIG. 3 is an enlarged detail view of FIG. 1, showing a proximal end of the adapter, including the electrical connector.

FIG. 4 is a partial schematic, transverse, cross-sectional view CS1 of the adapter of FIG. 1.

FIG. 5 is a partial schematic, transverse, cross-sectional view CS2 of the adapter of FIG. 1.

FIG. 6A is a partial schematic, perspective view of a balloon catheter parent before an adapter is attached to the distal end of the balloon catheter parent, and with the inflatable balloon represented as inflated for the purposes of illustration.

FIG. 6B is a partial schematic, perspective view of an adapter according to the present disclosure, attached to the distal end of a balloon catheter, and with the inflatable balloon represented as inflated for the purposes of illustration.

FIG. 7 is a schematic, perspective view of an adapter according to the present disclosure, attached to the distal end of a balloon catheter, and with a proximal electrical modular interface attached at the proximal end, forming an electrical modular catheter system. The balloon is represented as inflated for the purposes of illustration.

FIG. 8 is a schematic, perspective view of an adapter according to the present disclosure.

FIG. 9 is an enlarged detailed view of FIG. 8, showing part of a distal portion of the adapter, the attachment mechanism, and other features.

FIG. 10 is an enlarged detailed view of FIG. 8, showing the internal features and elements of a distal portion of the adapter.

FIG. 11 is a partial schematic, longitudinal view of an adapter according to the present disclosure. Break line symbols are utilized to reduce the size of the drawing for clarity.

FIG. 12 is a partial schematic, transverse, cross-sectional view CS3 of the adapter of FIG.

FIG. 13 is a partial schematic, transverse, cross-sectional view CS4 of the adapter of FIG.

FIG. 14 is a partial schematic, transverse, cross-sectional view CS5 of the adapter of FIG.

FIG. 15 is an enlarged detailed view of FIG. 8, showing the internal features and elements of a distal portion of the adapter.

FIG. 16 is a schematic, perspective view of an adapter according to an embodiment of the present disclosure.

FIG. 17 is an enlarged detailed view of FIG. 16, showing part of a distal portion of the adapter, the attachment mechanism, and other features.

FIG. 18 is a partial schematic, perspective view of an adapter according to the present disclosure, attached to the distal end of a balloon catheter. The balloon is represented as inflated for the purposes of illustration.

FIG. 19 is a partial schematic, longitudinal view of an adapter according to the present disclosure. Break line symbols are utilized to reduce the size of the drawing for clarity.

FIG. 20 is a partial schematic, transverse, cross-sectional view CS6 of the adapter of FIG. 19.

FIG. 21 is a partial schematic, transverse, cross-sectional view CS7 of the adapter of FIG. 19.

FIG. 22 is an example of a wiring schematic for use with an adapter according to the present disclosure.

FIG. 23 is an example of another wiring schematic for use with an adapter according to the present disclosure.

FIG. 24 is an alternate electrode configuration according to the present disclosure.

FIG. 25 is a partial schematic, transverse, cross-sectional view CS8 of the adapter of FIG. 24.

FIG. 26 is a partial schematic, perspective view of an adapter according to the present disclosure.

FIG. 27 is a partial schematic longitudinal views with partial cutaway cross-sections of an alternate electrode configuration according to the present disclosure.

FIG. 28 is a partial schematic longitudinal views with partial cutaway cross-sections of an alternate electrode configuration according to the present disclosure.

### DETAILED DESCRIPTION

In accordance with the present disclosure, an adapter may be constructed to have a proximal portion that interfaces with a medical device or parent module and a distal portion that modifies, augments, or extends the configuration or intended use of the medical device. As an example, the medical device may be a catheter. The adapter or adapter module is also a medical device and can be thought of as an accessory to the parent module medical device, augmenting the performance or functionality. In one aspect, an attachment mechanism of the adapter may secure the adapter to the distal end of the medical device catheter during use. The distal portion of the adapter may extend distally from the distal end of the catheter and is designed with features that expand, augment, or modify the configuration or intended use of the medical device catheter, such as with lithotripsy functionality as described further herein.

The proximal portion of the adapter may be designed to couple, such as through an interference fit, with an internal lumen of the medical device such that during subsequent use the adapter remains securely attached. The proximal portion may be additionally designed to be easily inserted into the internal lumen of a medical device. The proximal portion of the adapter may include an attachment mechanism, more completely described below, that provides securement between the adapter and medical device. The adapter and medical device comprise two modules of a modular medical device catheter system. The attachment mechanism allows an adapter module and a medical device module, also referred to as the parent module, to be combined as required by the physician or physician's staff in the operating room during a medical procedure to create a modular medical device catheter system. Varying combinations of adapter modules, or adapters and parent modules or parents, allows multiple variants of a medical device catheter to be flexibly created according to the dynamic needs and challenges of each patient and procedure. The modular medical device catheter system according to the present disclosure provides the physician with the benefit of flexibility to construct a medical device catheter of their choosing, combining structural, therapeutic, and diagnostic elements at the distal end for a specific procedural need. It also provides the hospital with inventory benefits, i.e. more medical device catheter variants from fewer inventory items or modules.

The medical device or parent module typically has a proximal end that remains outside the body of the patient and a distal end that goes inside the body of the patient. Examples of parent modules include but are not limited to: balloon catheters, stent delivery system catheters, transcatheter replacement valves and associated delivery catheters, stent graft delivery catheters, dissection repair catheters, atherectomy catheters, ablation catheters, aspiration catheters, and thrombectomy catheters.

An example of a suitable modular catheter system for use with the present disclosure is described in US Patent Publication Number 2020/0171295 by the inventor, published on Jun. 4, 2020, and hereby incorporated by reference in its entirety.

If the adapter module of a modular medical device catheter system includes an internal lumen, additional adapter modules can be added using this internal lumen to further add features, creating an enhanced modular medical device catheter, such as a parent plus a plurality of adapters. The modular arrangement allows a parent and adapter combination to become a parent in a new parent and adapter combination.

The adapter may also include conductors to transmit electrical signals from outside the patient body to the distal end of the parent device. One application of this may be an adapter with a distal portion that includes electrodes powered or activated in a manner similar to an electrophysiology catheter. The conductor in electrophysiology catheters are sometimes fine scale copper magnet wire, e.g. 35 gauge, or other polymer coated wire conductors, and similar conductors could be used in an electrophysiology adapter. Conductors may be housed inside the central tube, electrically connecting the distal portion of the adapter to outside the patient. The tube, wire or mandrel could extend proximally all the way out the proximal end of the target catheter or device.

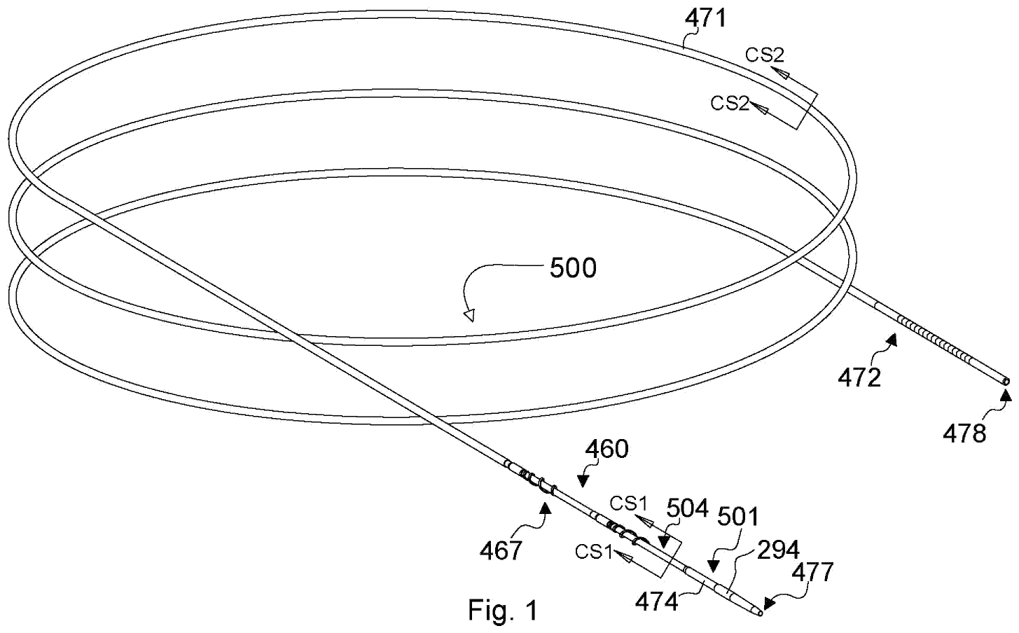

FIG. 1 is a schematic, perspective, view of an electrical adapter 500 according to an aspect of the present disclosure. Electrical adapter 500 includes a distal portion 501, which includes an electrical active element 294 and runway 474. Electrical adapter 500 includes a distal end 477 and a proximal end 478. Electrical adapter 500 includes a proximal portion 504 that incorporates an attachment mechanism 467 and elongate body 460. Electrical adapter 500 also includes a tubular extension 471 and electrical connector 472.

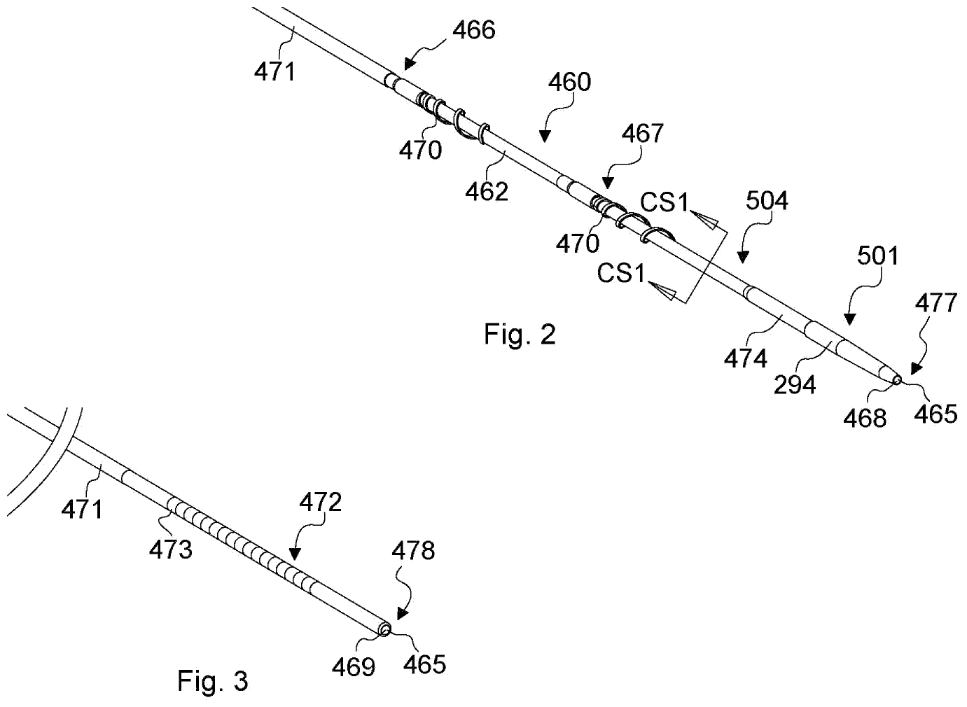

FIG. 2 is an enlarged detailed view of the proximal portion 504 of electrical adapter 500, distal portion 501 of electrical adapter 500, attachment mechanism 467, and elongate body 460. Attachment mechanism 467 includes elongated element or central tube 462 and interfacing elements 470. Elongate body 460 includes a tubular extension 471, extending from the proximal end 466 of central tube 462. The distal portion 501 includes a distal exit 468 for a central lumen 465 at the distal end 477 of the adapter 500.

FIG. 3 is an enlarged detailed view of the proximal end 478 of electrical adapter 500 showing a proximal exit 469 for a central lumen 465 at the proximal end 478 of adapter 500, tubular extension 471, and electrical connector 472 which includes ring electrical contacts 473.

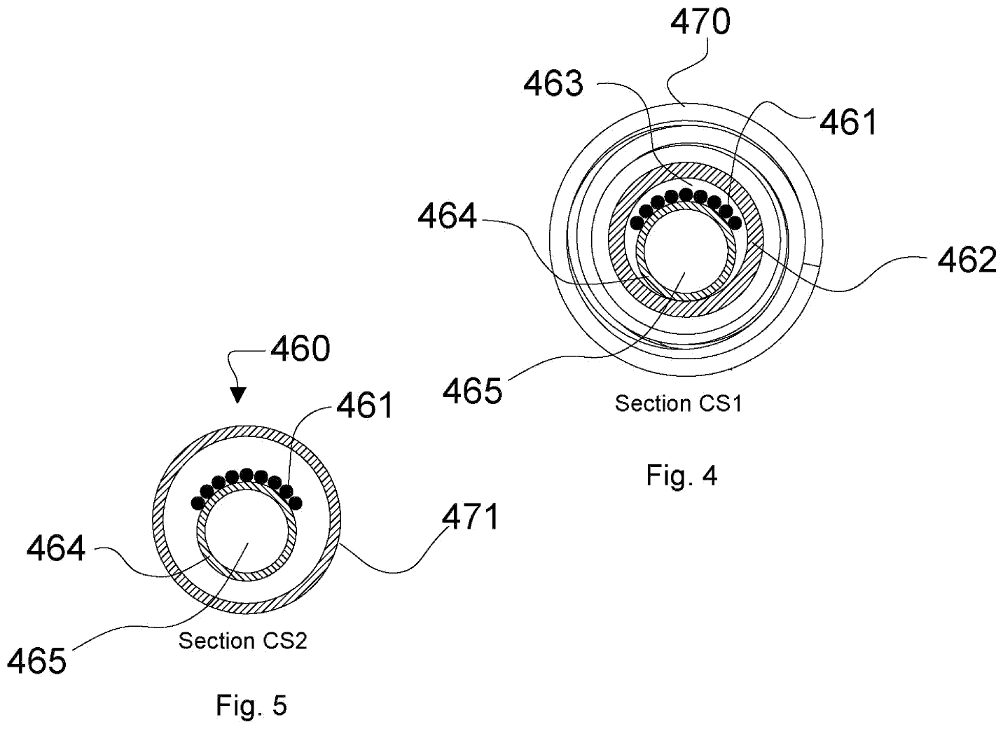

FIG. 4 is a partial schematic, transverse, cross-sectional view of electrical adapter 500 at CS1 as illustrated in FIG. 1 showing electrical conductors 461 and second central tube 464, that creates central lumen 465, within the lumen 463 of elongated element 462, as well as interfacing element 470 bonded to the outside of elongated element 462.

FIG. 5 is a partial schematic, transverse, cross-sectional view of electrical adapter 500 at CS2 as illustrated in FIG. 1 showing tubular extension 471 of the elongated body 460 which provides a lumen or conduit for both the electrical conductors 461 and second central tube 464, which creates a central lumen 465.

FIG. 1-5 show electrical adapter 500, which includes a distal portion 501 that may comprise, for example, electrically active elements 294 such as intravascular ultrasound (IVUS) transducers, lithotripsy electrodes, pressure sensors, imaging sensors, thermocouples, ablation electrodes, and other features requiring electrical signal transmission or electrical power. Electrical adapter 500 may also include a proximal portion 504 that incorporates an attachment mechanism 467 and elongate body 460. The elongate body 460 of electrical adapter 500 includes electrical conductors 461, for example, to facilitate electrical communication between the electrical connector 472 and electrodes described further herein. In this configuration, the conductors 461 extend proximally from the distal portion 501 of adapter 500 through the lumen 463 of the central tube or elongated element 462 but outside the lumen 465 of a second central tube 464 within the central tube 462. The second central tube 464 may be used by a physician as a guidewire lumen using over the wire techniques after the electrical medical device catheter system 600 is assembled.

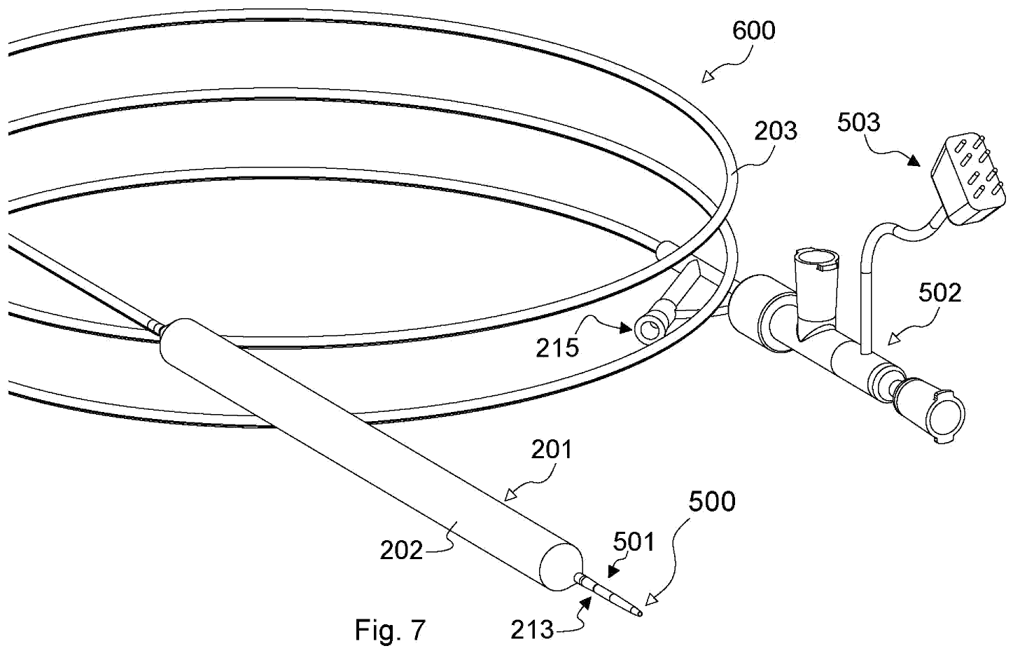

The second central tube 464 may also be omitted from the design, for example, if a guidewire lumen is not necessary, which may be the case for rapid exchange style configurations of the adapter 500. In the case where a central tube lumen 463 is not needed for a guidewire, the central tube lumen 463 could be used both as a passageway for conductors as well as an inflation lumen in alternate configurations of the distal portion 501 of the adapter 500, for example, where the adapter 500 includes a balloon to be inflated in-vivo. In either case, the second central tube 464 could extend proximal to or past the proximal end 219 of a medical device catheter 201 (shown in 6 and FIG. 7, for example). It may be advantageous for the proximal end 466 of central tube 462 to only extend far enough for the attachment mechanism 467 to incorporate compressible interfacing elements 470 to ensure secured coupling between the adapter 500 and a medical device catheter 201. The compressible interfacing elements 470 are designed to compress to interface with a lumen 211 at the distal end 213 of medical device catheter 201 to secure the electrical adapter 500 at the distal end 213 of medical device catheter 201. These compressible interfacing elements 470 are also described with reference to US Patent Publication Number 2020/0171295 by the inventor, hereby incorporated by reference in its entirety.

In an alternate embodiment, the proximal end 466 of central tube 462 could extend to a position proximal to a proximal end 219 of a medical device catheter 201. It may be advantageous when using the adapter 500 to have the conductors 461 bonded or attached to the outer surface of the second central tube 464. Alternatively heat shrink tubing, such as thin-walled polyester heat shrink tubing, could be used to hold the conductors 461 against the outer surface of the second central tube 462 in regions proximal to the proximal end 466 of central tube 462, creating a cohesive structure. Another alternative is to reflow a polymer jacket around the conductor 461 and second central tube 464 configurations in a manner similar to other catheter manufacturing techniques, such as guide catheter manufacturing. Another alternative is to incorporate a metallic or polymer spiral or coil around the length of the conductor 461, second central tube 464, and central tube 462 configuration in a manner similar to a conventional 0.035″ guidewire and provide the buckling stability of a guidewire.

FIG. 4 is transverse cross-sectional view at location “CS1” of FIG. 1 and FIG. 2, illustrating an example of a nine (9) conductor 461 configuration. The electrical conductors 461 may comprise standard round 42 AWG magnet wire, for example. It can be appreciated that the configuration, geometry, and number of electrical conductors can be tailored to the requirements of the electrically active elements of the adapter 500.

FIG. 5 is a transverse cross-sectional view at location “C52” of FIG. 1, illustrating elongate body 460 for adapter 500 which includes a tubular extension 471, extending from the proximal end 466 of central tube 462. Tubular extension 471 provides a conduit for both the electrical conductors 461 and second central tube 464.

The electrical conductors 461 can extend proximally from any electrically active elements 294 at distal portion 501 to a position proximal to the proximal end 219 of a medical device catheter 201, with or without central tube 462, second central tube 464, or tubular extension 471 also extending to a position proximal to the proximal end 219 of a medical device catheter 201. In an alternate embodiment of electrical adapter 500, electrically active elements could be positioned proximal to the attachment mechanism 467 instead of at the distal portion 501.

As illustrated in FIG. 1-5, the proximal end 478 of adapter 500 may comprise electrical connector 472 in electrical communication with the electrodes described further in the present disclosure. Connector 472 may comprise a ring electrical contact 473 for each conductor 461 used, for example, nine (9) ring electrical contacts 473 for each of the nine (9) electrical conductors 461. Second central tube 464 may include a distal exit 468 for lumen 465 at the distal end 477 of the adapter 500 and a proximal exit 469 at the proximal end 478 of adapter 500.

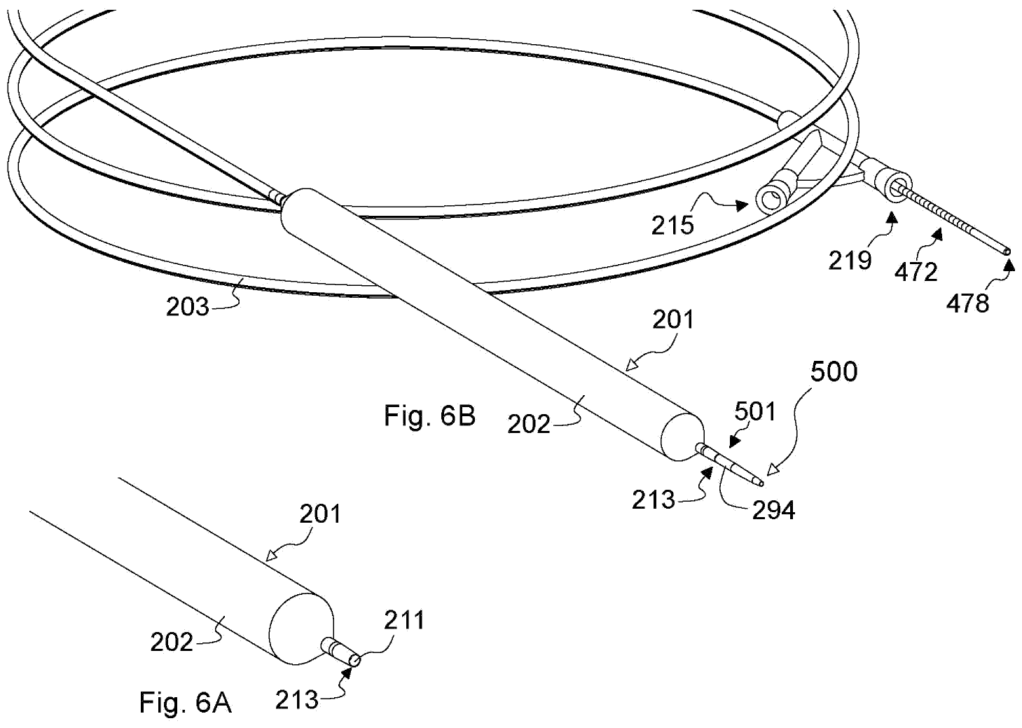

FIG. 6A is a partial schematic, perspective view of a balloon catheter or parent module 201, which is a medical device catheter, which includes a lumen 211 at the distal end 213, before electrical adapter 500 is attached to the distal end 213 of the balloon catheter 201, and with inflatable balloon 202 represented as inflated for the purposes of illustration.

FIG. 6B is a partial schematic, perspective view of electrical adapter 500, according to an aspect of the present disclosure, attached to the distal end 213 of a balloon catheter 201, and with the inflatable balloon 202 represented as inflated for the purposes of illustration. As shown, the electrically active element 294 of distal portion 501 is distal to the distal end 213 of balloon catheter 201. The proximal end 478 of electrical adapter 500 and electrical connector 472 are proximal to the proximal end 219 of balloon catheter 201. Balloon catheter 201 includes a catheter shaft 203 to connect inflatable balloon 202 to a fitting assembly 215.

FIG. 7 is a schematic, perspective view of an assembled electrical modular catheter system 600 according to an aspect of the present disclosure. Assembled electrical modular catheter system 600 is a combination of medical device catheter 201 (also known as the parent module), electrical adapter 500, and proximal module 502. Proximal module 502, includes an electrical connector interface 503 and is attached to the proximal end 219 of fitting assembly 215 at the proximal end of the balloon catheter 201. The inflatable balloon 202 of balloon catheter 201 is represented as inflated for the purposes of illustration.

FIG. 6A and FIG. 6B illustrate the features of medical device balloon catheter 201 which includes a distal end 213 and proximal end 219. The balloon catheter 201 includes an inflatable balloon 202 positioned near the distal end 213. The inflatable balloon 202 is connected to a fitting assembly 215 near the proximal end 219 of medical device balloon catheter 201 by a catheter shaft 203. The catheter shaft 203 is typically a long tube with one or more lumens, at least one lumen 211 has an opening near the distal end 213.

FIG. 6B also illustrates electrical adapter 500 after it has been secured to medical device balloon catheter 201. Electrical adapter 500 is attached to medical device balloon catheter 201 by inserting the proximal end of adapter 478 into the distal end 213 of a lumen 211 of balloon catheter 201 until the attachment mechanism 467 has secured the adapter 500 to the balloon catheter 201. Interfacing elements 470, of the attachment mechanism 467, are attached or otherwise bonded to the elongated element 462 and configured to secure the electrical adapter 500 to a medical device catheter. Balloon catheter 201 is shown with the inflatable balloon 202 in an inflated state for illustration purposes but would normally be in a deflated state during the attachment of adapter 500 to balloon catheter 201. Alternatively, electrical adapter 500 could be attached to any other appropriate medical device catheter 201, for example a stent delivery system. Balloon catheter 201 may also include a fitting assembly 215 near the proximal end 219 of medical device balloon catheter 201 that includes a port to inflate the balloon and a port for “over-the-wire” guidewire access. The lumen 211 of a balloon catheter 201 is typically available to be used with a guidewire during a minimally invasive medical procedure. As described previously, electrical adapter 500 distal portion 501 may comprise, for example, electrically active elements 294, near the distal end 213 of the parent medical device catheter 201.

FIG. 7 illustrates the electrical adapter 500 after it has been secured to a medical device balloon catheter 201 and after a proximal module 502 has been attached to the proximal end 219 of the balloon catheter 201 and the proximal end 478 of electrical adapter 500. Proximal module 502 may include an electrical connector interface 503 to provide an electrical connection between the ring electrical contacts 473 of electrical connector 472 and a user interface or equipment for the electrically active adapter 500.

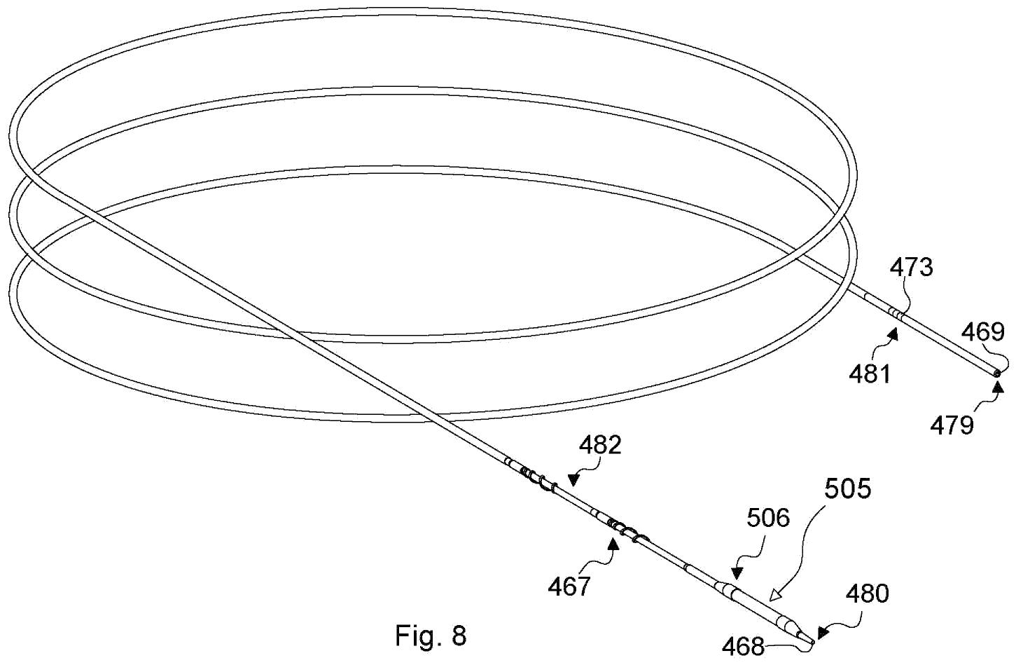

FIG. 8 is a schematic, perspective view of an over-the-wire (OTW) intravascular lithotripsy (IVL) adapter 505 according to an aspect of the present disclosure. OTW IVL adapter 505 includes a distal portion 506, which includes a distal exit 468 for a central lumen 465 at a distal end 480. OTW IVL adapter 505 also includes an attachment mechanism 467, elongate body 482, proximal end 479, proximal electrical connector 481, which includes ring electrical contacts 47. OTW IVL adapter 505 also includes proximal exit 469 at the proximal end 478 of OTW IVL adapter 505.

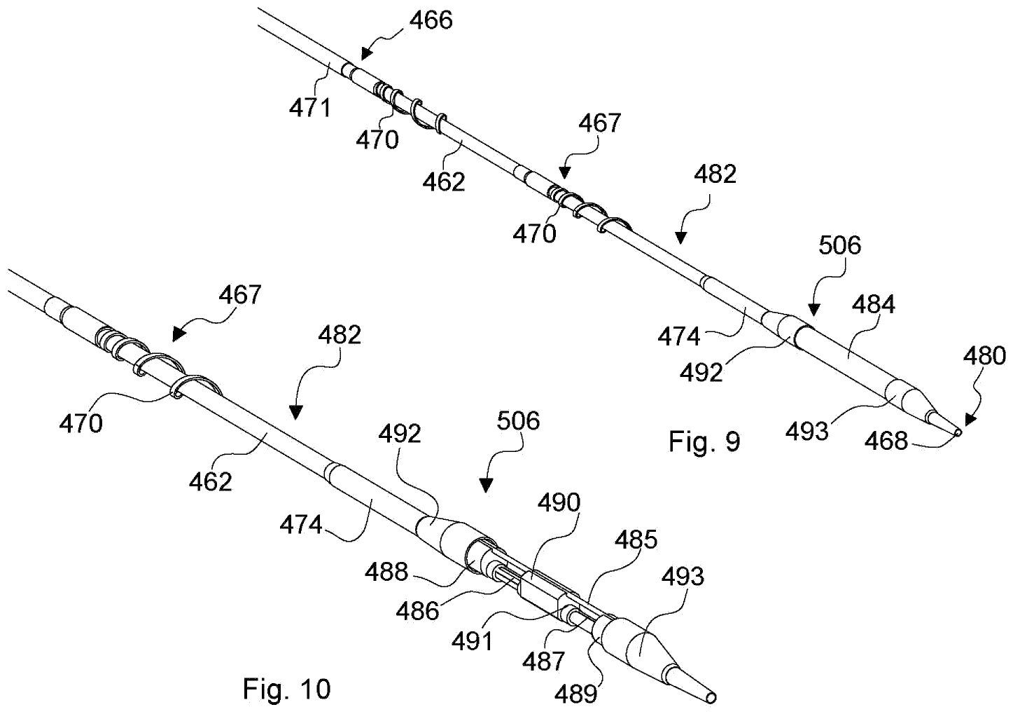

FIG. 9 is an enlarged detailed view, showing distal portion 506 of over-the-wire (OTW) intravascular lithotripsy (IVL) adapter 505, the attachment mechanism 467, and tubular extension 471 among other features. Distal portion 506 has a distal end 480 and includes runway 474, an outer tube 484, and the proximal and distal jacket or coverings 492 and 493 at the ends of outer tube 484. Attachment mechanism 467 includes elongated element 462 and interfacing elements 470. Elongated element 462 has a proximal end 466. FIG. 9 also shows elongate body 482.

FIG. 10 is an enlarged detailed view, showing distal portion 506 of over-the-wire (OTW) intravascular lithotripsy (IVL) adapter 505, like FIG. 9, but with outer tube 484 not shown to illustrate a cavitation bubble chamber 491, first electrode 486, second electrode 487, intermediate electrode 485, chamber separator 490, proximal plug 488, and distal plug 489.

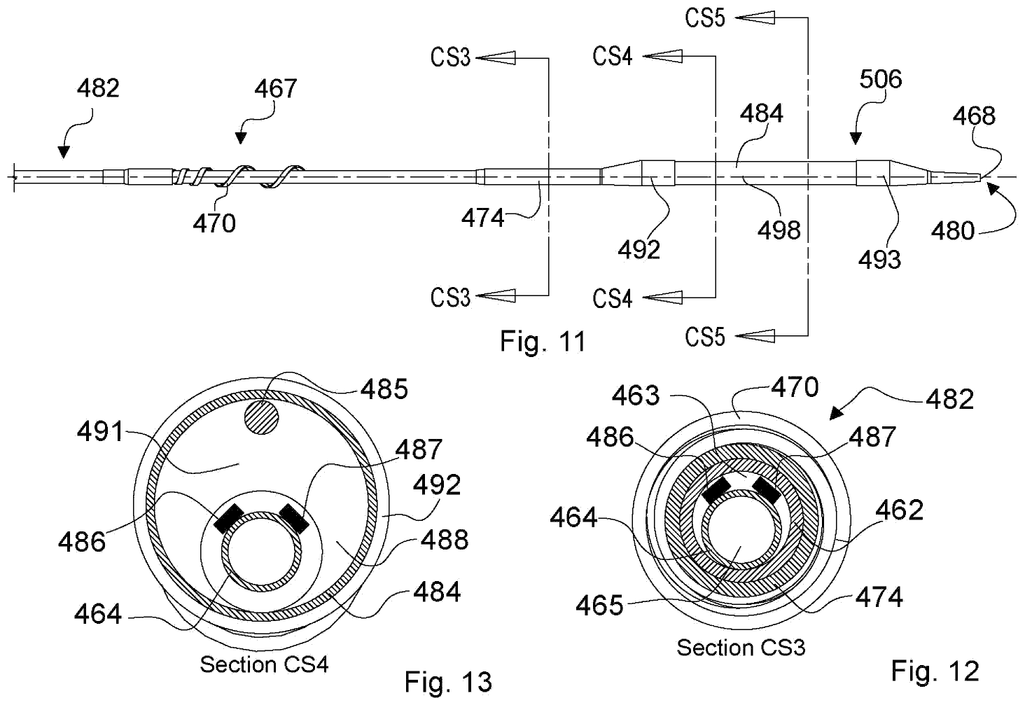

FIG. 11 is a partial schematic, longitudinal view of over-the-wire (OTW) intravascular lithotripsy (IVL) adapter 505 according to an aspect of the present disclosure. OTW IVL adapter 505 includes a distal portion 506, which includes a distal exit 468 for a central lumen 465 at a distal end 480 and includes runway 474, an outer tube 484, and the proximal and distal jacket or coverings 492 and 493 at the ends of outer tube 484. OTW IVL adapter 505 also includes an attachment mechanism 467 and elongate body 482. FIG. 11 also illustrates long or longitudinal axis 498 of the adapter 505 and cavitation bubble chamber 491.

FIG. 12 is a partial schematic, transverse, cross-sectional view of OTW IVL adapter 505 at CS3 as illustrated in FIG. 11 showing elongate body 482 which includes first electrode 486, second electrode 487, second central tube 464, that creates central lumen 465, within the lumen 463 of elongated element 462. Also shown are interfacing element 470 bonded to the outside of elongated element 462, and runway 474.

FIG. 13 is a partial schematic, transverse, cross-sectional view of OTW IVL adapter 505 at CS4 as illustrated in FIG. 11 showing outer tube 484, first electrode 486, second electrode 487, intermediate electrode 485, second central tube 464, cavitation bubble chamber 491, proximal plug 488, and proximal jacket or covering 492.

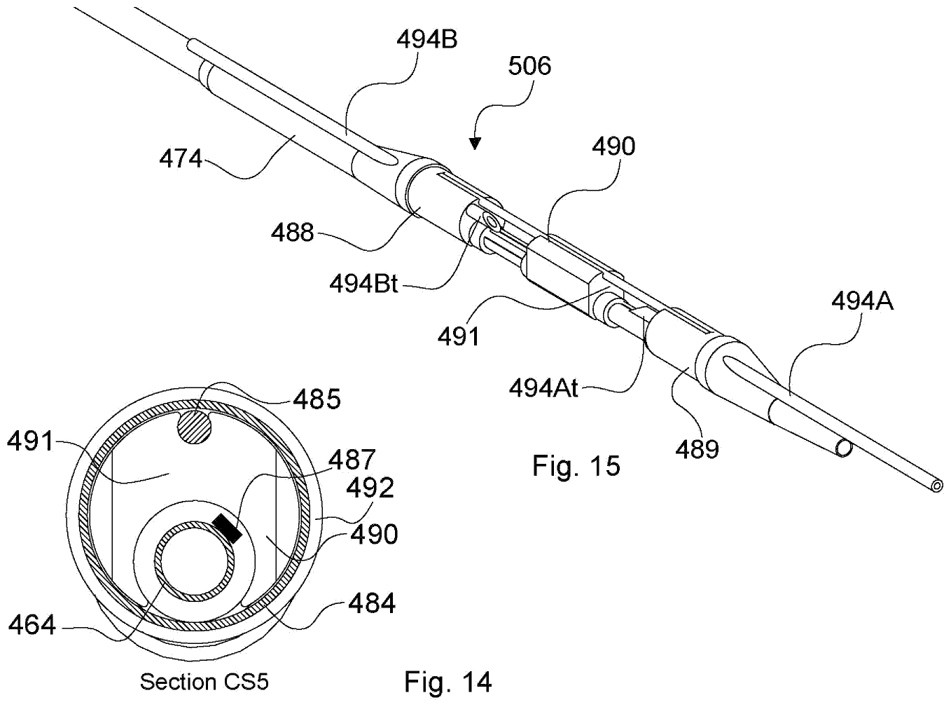

FIG. 14 is a partial schematic, transverse, cross-sectional view of OTW IVL adapter 505 at CS5 as illustrated in FIG. 11 showing outer tube 484, second electrode 487, intermediate electrode 485, second central tube 464, cavitation bubble chamber 491, chamber separator 490, and proximal jacket or covering 492

FIG. 15 is an enlarged detailed view of distal portion 506 of OTW IVL adapter 505 like FIG. 9, but with outer tube 484, proximal jacket or covering 492, and distal jacket or covering 493 not shown to illustrate a cavitation bubble chamber 491, chamber separator 490, proximal plug 488, and distal plug 489. FIG. 15 also illustrates two needles 494A and B, which may be used to puncture the proximal plug 488 and distal plug 489, forming the boundary of the cavitation bubble chamber 491 along with the outer tube 484 (not shown), with the sharp tip of the needles 494At and/or 494Bt, penetrating and entering the cavitation bubble chamber 491. FIG. 8-10 illustrate an example of an over-the-wire (OTW) intravascular lithotripsy

(IVL) adapter 505, with a distal end 480 and proximal end 479. OTW IVL adapter 505 is similar to the previously described electrical adapter 500 in that it comprises an elongate body 482, similar to elongate body 460, and attachment mechanism 467. OTW IVL adapter 505 also includes a distal portion 506 with a cavitation bubble chamber 491 within the body of the distal portion 506 for containing a cavitation solution. In one example, the cavitation bubble chamber 491 is filled with cavitation solution, typically with a conductivity solution below 20 micro-siemens per centimeter (0/cm) during the manufacturing process. Viable cavitation solutions may include a 0.8M saccharose solution or deionized water, for example. Instead of filling the cavitation bubble chamber 491 during manufacturing, in an alternative embodiment, the cavitation bubble chamber 491 can be filled with a cavitation solution during a minimally invasive or endovascular procedure, for example, tableside in an operating room prior to inserting the adapter 505 and parent catheter 201 or combined modular system into the patient.

As shown with further reference to features of FIG. 11 and FIG. 12, the lumen 463 of the central tube 462 of the elongate body 482 could be used to fill the cavitation bubble chamber 491 with an appropriate solution during a procedure.

As illustrated in FIG. 9-11, 13-15, the cavitation bubble chamber 491 is formed by an outer tube 484 located at distal portion 506 (note FIG. 10 illustrates distal portion 506 of adapter 505 without the outer tube 484 to show the internal features and elements relating to the cavitation bubble chamber 491). Additionally, the outer tube 484 is enclosed by a proximal plug 488 and a distal plug 489. The proximal plug 488 and distal plug 489 can be made from a polymer, typically through a molding manufacturing process or an extrusion process, with secondary reflow or bonding processes to enclose the proximal and distal ends of the outer tube 484 thereby creating the cavitation bubble chamber 491. Additionally, inside the outer tube 484 is a center chamber separator 490 to separate the chamber into two spaces where a cavitation bubble can be created between two distinct electrode sets, first electrode 486 and intermediate electrode 485, and second electrode 487 and intermediate electrode 485. The chamber separator 490 can also serve to support the center of the intermediate electrode 485, while the proximal plug 488 and distal plug 489 support the ends of the intermediate electrode 485.

In the example illustrated in FIG. 11 and the transverse cross-sectional views of FIG. 12-14, the first electrode 486 and intermediate electrode 485, and second electrode 487 are illustrated as wires of various cross sections running parallel to each other along the long or longitudinal axis 498 of the adapter 505 and cavitation bubble chamber 491. the second electrode 487 and first electrode 486 may be configured as flat wires with a rectangular cross section, where the intermediate electrode 485 may be configured as a round wire, with a circular cross section. Other cross-sectional shapes could be useful, such as electrode wire with triangular cross sections. An advantage of this parallel electrode configuration is that the arcing or spark generation between the electrodes can happen anywhere along the parallel lengths where the electrodes are mutually exposed (do not have electrical insulating coatings or covering). This may allow more cycles of arcing or spark generation because as the electrode wears with repeated arcing cycles the arcing can migrate to a fresh wire location farther along the parallel electrode wire set length. These electrodes may suitably be manufactured from copper, graphite, tungsten, stainless steel or other appropriate conducting materials. If the cavitation bubble chamber 491 is filled with the cavitation solution during the manufacturing process and will be in contact with the electrodes 487, 486 or 485, it may be advantageous to coat the conducting material with gold or other protective coating to minimize oxidation during an extended period of storage, such as during the shelf life of the product. If conductive wire is used as electrode 487 and 486, this wire can extend through the elongated body 482 to the ring electrical contacts 473 in electrical connector 481 of electrical adapter 505, to provide electrical continuity for communication with high voltage pulse generator 457. Alternatively, the electrodes 487 and 486 can be electrically connected to other electrical conductors 461 within or proximal to the cavitation bubble chamber 491 which are then electrically connected to the appropriate ring electrical contacts 473 in electrical connector 481 of adapter 505, such as shown in FIG. 8.

One method to fill the cavitation bubble chamber 491 with a cavitation solution is illustrated in FIG. 15 (note FIG. 15 illustrates distal portion 506 of OTW IVL adapter 505 without the outer tube 484, or the proximal and distal jacket or coverings 492 and 493 such as shown in FIG. 10. This is done to show the internal features and elements related to the cavitation bubble chamber 491). As shown, two needles 494A and B may be used to puncture the proximal plug 488 and distal plug 489 that form the boundary of the cavitation bubble chamber 491 along with the outer tube 484 (not shown), with the sharp tip of the needles 494At and/or 494Bt, penetrating and entering the cavitation bubble chamber 491. The cavitation solution may then be injected through the lumen of one or both of the needles 494A, B to fill the cavitation bubble chamber 491. It may be advantageous to inject the cavitation solution through one of the lumens of the needles 494A or B, while the other needle allows entrapped air to escape to enable a more complete filling of the cavitation bubble chamber 491. After the cavitation bubble chamber 491 is filled with the cavitation solution, it may be appropriate or necessary to cover the puncture sites in the proximal plug 488 and distal plug 489 with a proximal jacket or covering 492 and a distal jacket or covering 493 to seal the puncture sites (such as also shown in FIG. 9 and FIG. 10), ensuring the cavitation solution does not leak from the cavitation bubble chamber 491. The proximal jacket 492 and distal jacket 493 could be formed from a polymer and bonded, welded or attached to the distal portion 506. Alternatively, it may be advantageous to laser weld the puncture sites to seal the cavitation bubble chamber 491, among other techniques as may be appreciated in the art.

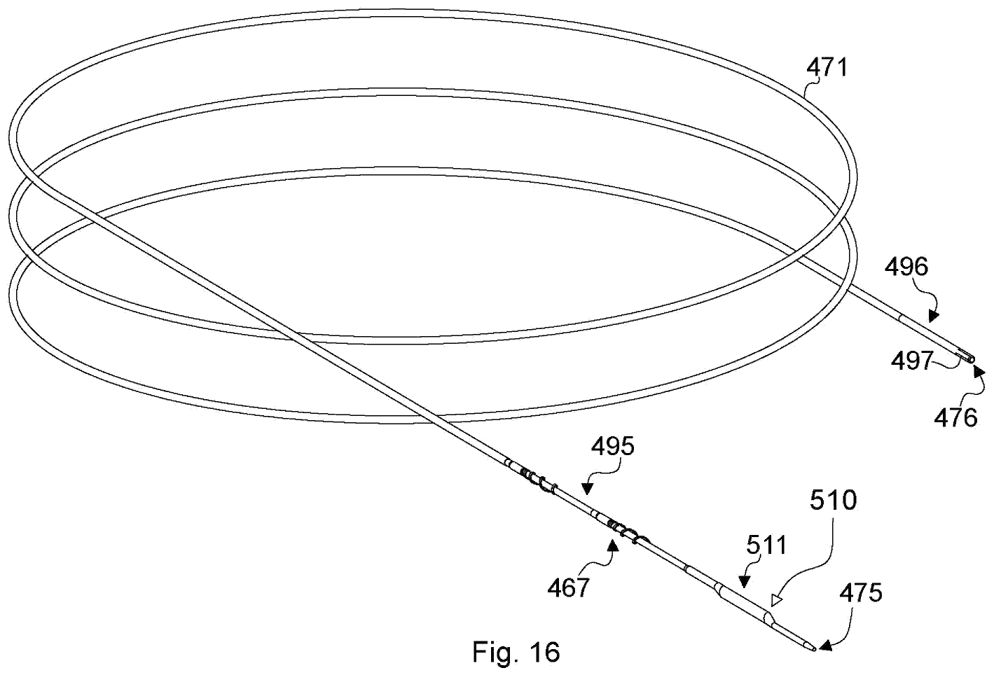

FIG. 16 is a schematic, perspective view of a rapid exchange (RX) intravascular lithotripsy (IVL) adapter 510 according to an aspect of the present disclosure. RX IVL adapter 510 includes a distal portion 511. RX IVL adapter 510 includes a distal end 475 and a proximal end 476. RX IVL Adapter 510 incorporates an attachment mechanism 467 and elongate body 495. RX IVL Adapter 510 also includes a tubular extension 471 and electrical connector 496, which includes tab electrical contacts 497.

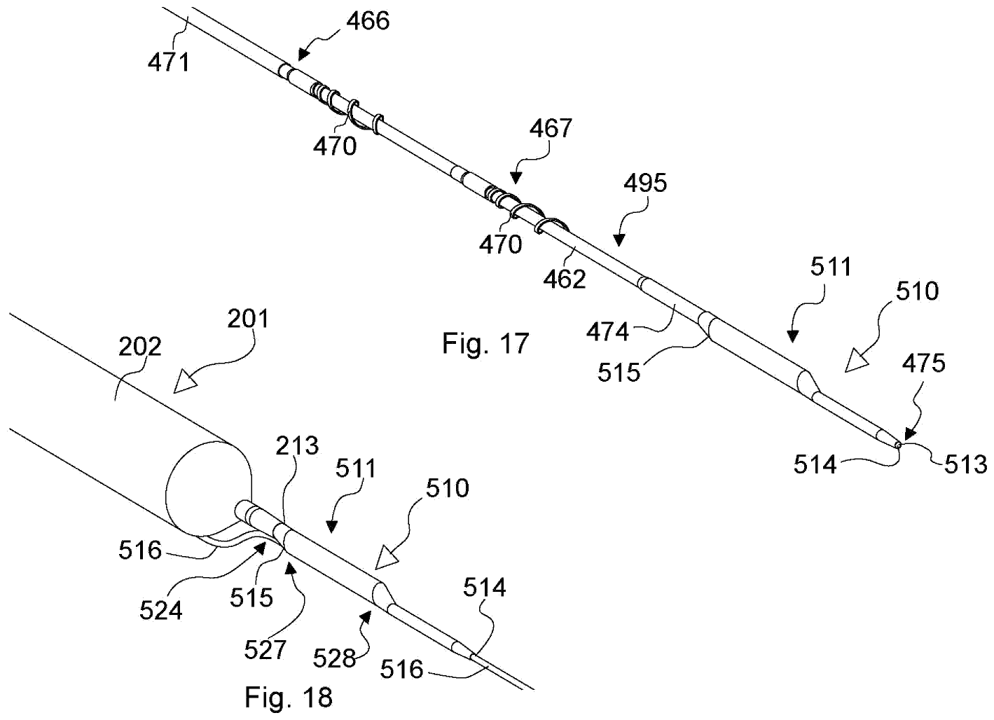

FIG. 17 is an enlarged detailed view of a rapid exchange (RX) intravascular lithotripsy (IVL) adapter 510 according to an aspect of the present disclosure illustrated in FIG. 16, showing distal portion 511 of RX IVL adapter 510, the attachment mechanism 467, elongate body 495, distal end 475, and proximal end 466 of elongated element, also known as central tube 462. Distal portion 511 includes rapid exchange lumen 513 with a distal end 514 and a proximal end 515, and runway 474. Attachment mechanism 467 includes interfacing elements 470 and elongated element 462.

FIG. 18 is a partial schematic, perspective view of a rapid exchange (RX) intravascular lithotripsy (IVL) adapter 510 according to an aspect of the present disclosure, attached to a distal end 213 of a balloon catheter 201, where the inflatable balloon 202 is represented as inflated for the purposes of illustration, and a guidewire 516 is passing through distal end 514 and proximal end 515 of rapid exchange lumen 513 (illustrated in FIG. 17 and FIG. 21). Also illustrated in FIG. 18 is junction 524 between the distal portion 511 and distal end 213 of balloon catheter also known as parent module 201. Distal portion 511 of RX IVL adapter 510 includes cavitation bubble chamber 520 (illustrated in FIG. 21) which has a distal end 528 and a proximal end 527.

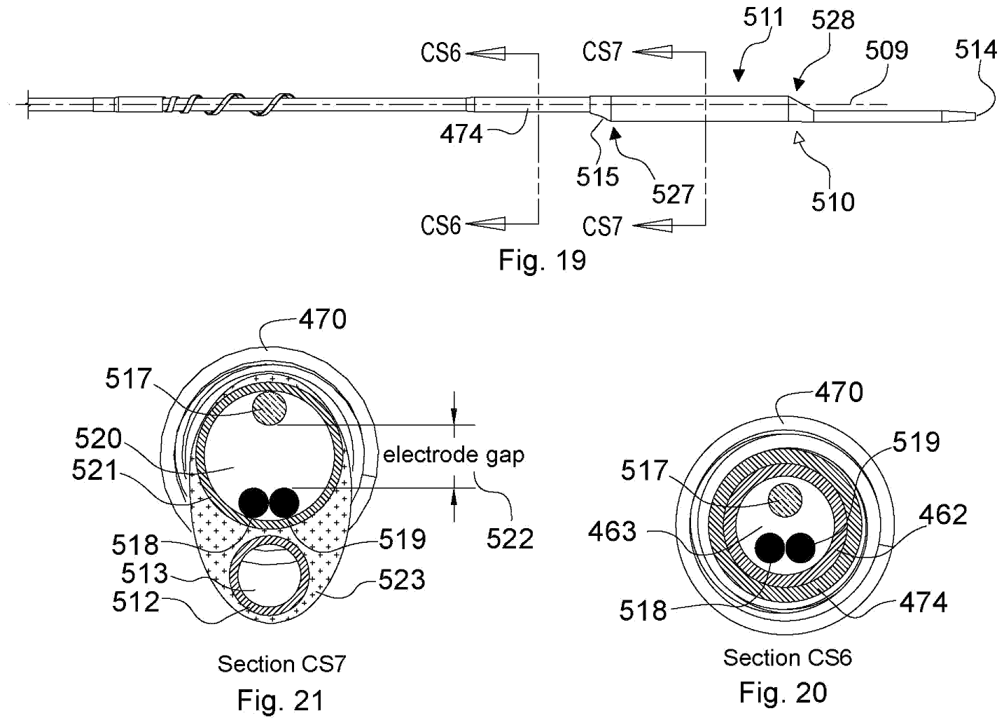

FIG. 19 is a partial schematic, longitudinal view of rapid exchange (RX) intravascular lithotripsy (IVL) adapter 510 according to an aspect of the present disclosure illustrated in FIG. 16, showing distal portion 511 of RX IVL adapter 510, Distal portion 511 includes rapid exchange lumen 513 (illustrated in FIG. 17 and FIG. 21) with a distal end 514 and a proximal end 515, runway 474, cavitation bubble chamber 520 (illustrated in FIG. 21) which has a distal end 528 and a proximal end 527. FIG. 19 also shows longitudinal or long axis 509 of the RX IVL adapter 510.

FIG. 20 is a partial schematic, transverse, cross-sectional view of RX IVL adapter 510 at CS6 as illustrated in FIG. 19 showing lumen 463 of elongated element 462, a first powered electrode 518, a second powered electrode 519, a ground electrode 517. Also shown is interfacing element 470, and runway 474.

FIG. 21 is a partial schematic, transverse, cross-sectional view of RX IVL adapter 510 at CS7 as illustrated in FIG. 19 showing cavitation bubble chamber 520, which is also the lumen of a cavitation bubble tube 521, a first powered electrode 518, a second powered electrode 519, a ground electrode 517, electrode gap 522 between electrodes, rapid exchange lumen 513 formed by a rapid exchange tube 512 surrounded by a polymer body 523. Also shown is interfacing element 470.

FIG. 16-21 illustrate another example intravascular lithotripsy (IVL) adapter 510 according to the present disclosure. Adapter 510 comprises a distal portion 511, an elongate body 495 similar to 460 described previously, attachment mechanism 467 and electrical connector 496 with tab electrical contacts 497. Electrical conductors 461 electrically connect the three (3) tab contacts 497 on electrical connector 496 with the three (3) electrodes in the cavitation bubble chamber 520, a first powered electrode 518, a second powered electrode 519, and a ground electrode 517. Rapid exchange (RX) intravascular lithotripsy (IVL) adapter 510 has a distal end 475 and a proximal end 476. Distal portion 511 of RX IVL adapter 510 includes a rapid exchange lumen 513 (shown in FIG. 21) with a distal end 514 and a proximal end 515, the proximal end 515 is distal to the distal end 213 of the parent medical device catheter 201 (such as shown in FIG. 18), after the RX IVL adapter 510 has been attached to the distal end of the medical device catheter 201 by inserting the proximal end 476 of adapter 510 into a lumen 211 at the distal end 213 of medical device catheter 201. The distal portion 511 of RX IVL adapter 510 includes a runway 474 (also shown with reference to FIGS. 9-11 and 17-19). After attaching the RX IVL adapter 510 to parent module (balloon catheter) 201 a portion of the runway 474 fits within a lumen 211 at the distal end 213 of parent module 201. Typically, runway 474 is smaller than the lumen 211 at the distal end 213 of parent module 201 and is comprised of a polymer bonded or attached to the central tube 462. A purpose of the runway 474 is to provide a robust transition or junction 524 between the distal portion 511 of RX IVL adapter 510 and distal end 213 of parent module (balloon catheter) 201. The runway 474 would be designed to minimize kinking or buckling at the junction 524 between the distal portion 511 and distal end 213 of the parent module 201. The design of the runway 474 could include stainless steel braiding or higher durometer polymers to aid in providing a stable junction 524, for example.

The rapid exchange lumen 513, shown in FIG. 21, is designed through the choice of geometry and material to function as a rapid exchange lumen 513 for a guidewire 516 (shown in FIG. 18) to be used during a medical procedure. The rapid exchange lumen 513 could be formed by a separate rapid exchange tube 512 surrounded by a polymer body 523 (shown, for example, in FIG. 21). For example, a suitable rapid exchange tube 512 could be a thin walled, approximately 0.002″ to 0.001″, polyimide tube.

As shown in FIG. 17 to FIG. 21, the distal portion 511 of RX IVL adapter 510 also comprises a cavitation bubble chamber 520, which is also the lumen of a cavitation bubble tube 521. The cavitation bubble chamber 520 can be filled with a cavitation solution similar to cavitation bubble chamber 491 described previously. As illustrated, Cavitation bubble chamber 520 has a distal end 528 and a proximal end 527. Cavitation bubble chamber 520 can also include an opening at the distal end 528 to facilitate filling the cavitation bubble chamber 520 with a cavitation solution by allowing any entrapped air bubbles or vapor bubbles to escape. Within the cavitation bubble chamber 520 are three (3) electrodes, including a first powered electrode 518, a second powered electrode 519, and a ground electrode 517. The three (3) electrodes 517, 518, and 519 are illustrated as wires of round cross sections running parallel to each other along the longitudinal or long axis 509 of the adapter 510 and cavitation bubble chamber 520. The proximal end 515 of the rapid exchange lumen 513 is just proximal to the proximal end 527 of the cavitation bubble chamber 520. Alternatively, the proximal end 515 of the rapid exchange lumen 513 could be located anywhere between the distal end 528 of the cavitation bubble chamber 520 and the proximal end 527 of the cavitation bubble chamber 520. It may be advantageous to construct the distal portion 511 of RX IVL adapter 510 configured with the proximal end 515 of the rapid exchange lumen 513 distal to the distal end 528 of the cavitation bubble chamber 520.

In this configuration, the rapid exchange lumen 513 would not have a portion running parallel to, or side by side with, the cavitation bubble chamber 520, as shown in FIG. 21, but could be characterized as a serial configuration, meaning the rapid exchange lumen 513 is more in line with cavitation bubble chamber 520. An advantage of the serial configuration would be a lower profile distal portion 511 with the drawback or tradeoff of a potentially longer distal portion 511.

The first powered electrode 518 and the second powered electrode 519 may also have an insulated coating that has been selectively removed or selectively applied such that a spark and cavitation plasma bubble 526 will be created across the electrode gap 522 at particular, or controlled uninsulated portions or locations along the length of the cavitation bubble chamber 520.

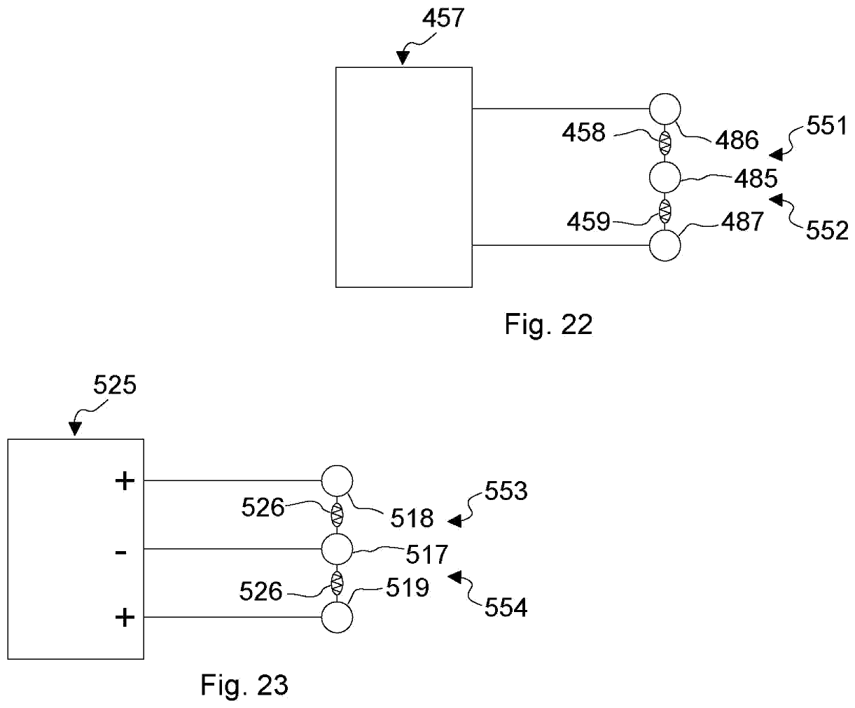

FIG. 22 illustrates an example of a wiring circuit schematic suitable for use with over-the-wire (OTW) intravascular lithotripsy (IVL) adapter 505 according to an aspect of the present disclosure. FIG. 22 shows a high voltage pulse generator 457 which creates cavitation bubble 458 and cavitation bubble 459 by serially applying a high voltage potential difference between a first electrode set 551, the first electrode 486 and intermediate electrode 485, as well as between a second electrode set 552, intermediate electrode 485 and second electrode 487.

FIG. 23 illustrates an example of wiring circuit schematic suitable for use with rapid exchange (RX) intravascular lithotripsy (IVL) adapter 510 according to an aspect of the present disclosure. As shown in FIG. 23 a high voltage pulse generator 525 creates an arc or spark within the cavitation solution at the electrode gap 522 between the parallel lengths of the first powered electrode 518 and the ground electrode 517 as well as the second powered electrode 519 and ground electrode 517 in cavitation bubble chamber 520, which in turn creates cavitation bubbles 526 by applying parallel high voltage potential difference between a first electrode set 553, the first powered electrode 518 and the ground electrode 517, as well as between a second electrode set 554, the second powered electrode 519 and ground electrode 517.

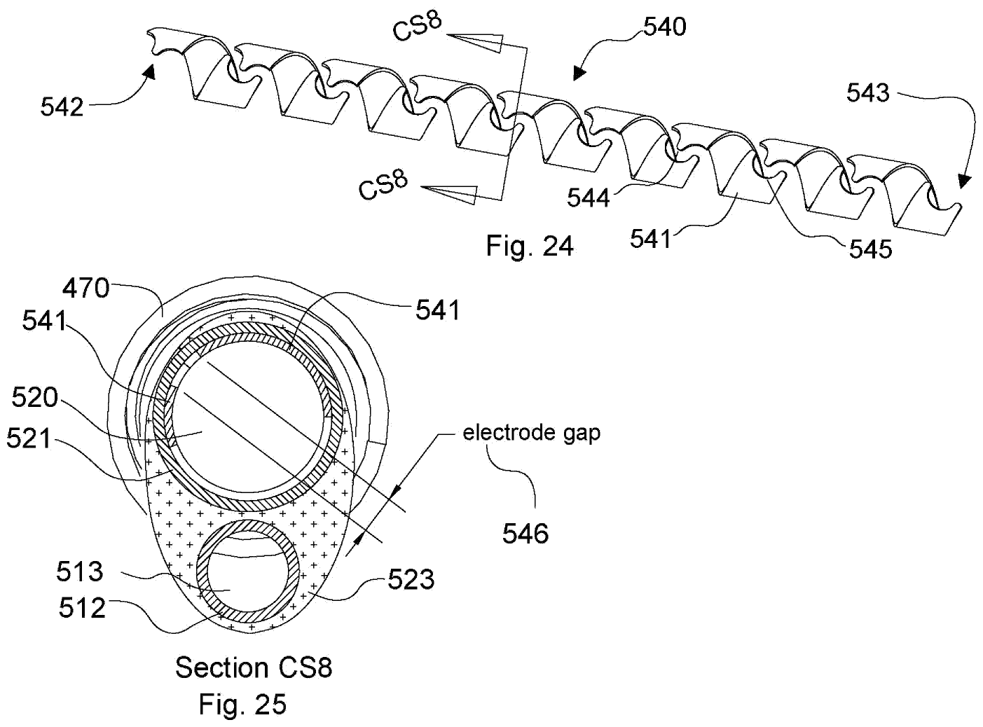

FIG. 24 illustrates a tubular electrode assembly 540 that could be incorporated into intravascular lithotripsy adapters according to an aspect of the present disclosure. Tubular electrode assembly 540 includes a series of tubular electrode elements 541 having a proximal end 544 and distal end 545 arranged in an end-to-end fashion, where the tubular electrode assembly 540 has a distal end 543 and proximal end 542.

FIG. 25 is a partial schematic, transverse, cross-sectional view of a RX IVL adapter similar to RX IVL adapter 510. The cross-sectional view is like that of FIG. 21 showing section CS7 as illustrated in FIG. 19, but showing a cross-sectional view of a RX IVL adapter with tubular electrode assembly 540 at a location CS8 of FIG. 24. FIG. 25 illustrates tubular electrode elements 541 assembled in a cavitation bubble tube 521 forming cavitation bubble chamber 520, and electrode gap 546 between adjacent tubular electrode elements 541. FIG. 25 illustrates the other features, rapid exchange lumen 513 formed by a rapid exchange tube 512 surrounded by a polymer body 523, and interfacing element 470.

FIG. 24 and FIG. 25 illustrate an example of a suitable electrode configuration according to the present disclosure. In this example a series of tubular electrode elements 541 are arranged end to end, into a tubular electrode assembly 540. As shown in the example of FIG. 24, nine (9) tubular electrode elements 541 are arranged in a series forming the tubular electrode assembly 540 having eight (8) electrode gaps 546. In this example, the tubular electrode element 541 can be manufactured by laser cutting the spiral shape from tubular stock of an appropriate material with the required diameter and wall thickness. The electrode gap 546 is formed between the proximal end 544 of a tubular electrode element 541 and the distal end 545 of an adjacent tubular electrode element. As an alternative to the spiral shape of the tubular electrode element 541, the shape could be a circumferential ring, where an appropriate electrode gap is configured between adjacent circumferential ring electrode elements. The tubular electrode element 541 at the proximal end 542 of the tubular electrode assembly 540 is electrically connected to one side of a high voltage pulse generator 457 (such as shown in FIG. 22) and the other electrical side of the high voltage pulse generator is electrically connected to the tubular electrode element 541 at the distal end 543 of the tubular electrode assembly 540. When an appropriate high voltage pulse is applied, a cavitation bubble will be created at each of the eight (8) electrode gaps 546. The tubular electrode assembly 540 could be incorporated into a distal portion of an adapter similar to distal portion 511 of adapter 510 described previously, but wherein the tubular electrode assembly 540 forms the cavitation bubble chamber 520. Cross-sectional view CS8 of FIG. 25 illustrates the adapter incorporating tubular electrode assembly 540 similar to RX IVL adapter 510 and the cross sectional view CS7 of FIG. 21 previously described, where the section arrows of FIG. 24 show approximate location of section CS8 of adapter 510 incorporating tubular electrode assembly 540. Electrode pair configurations, or electrode sets could include pairing a tubular electrode element with a wire or other electrode element.

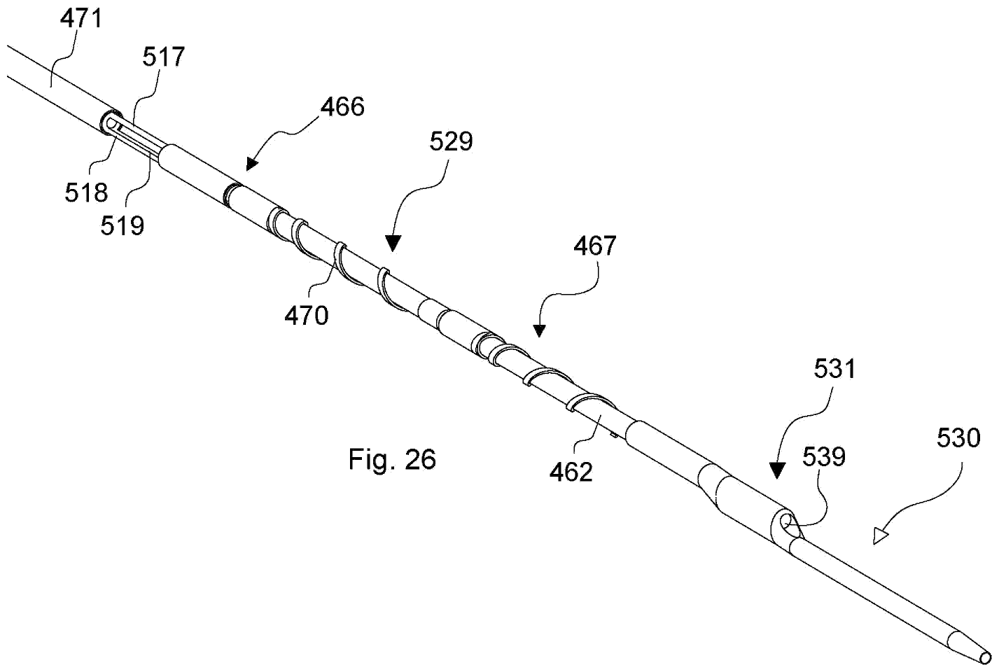

FIG. 26 is partial schematic view of an intravascular lithotripsy (IVL) adapter 530 according to an aspect of the present disclosure, showing distal portion 531 and proximal portion 529 of IVL adapter 530, attachment mechanism 467, and proximal end 466 of elongated element, also known as central tube 462. Distal portion 531 can include an opening 539 at the distal end to facilitate filling with a cavitation solution by allowing any entrapped air bubbles or vapor bubbles to escape. Proximal portion 529 includes attachment mechanism 467 which includes interfacing elements 470 and elongated element 462, three (3) electrodes, a first powered electrode 518, a second powered electrode 519, and a ground electrode 517, and tubular extension 471. Three (3) electrodes, a first powered electrode 518, a second powered electrode 519, and a ground electrode 517 are proximal to proximal end 466 of elongated element, also known as central tube 462. In another example as illustrated in FIG. 26, electrode configurations similar to that illustrated in adapter 505 and 510 previously described could be positioned proximal to the attachment mechanism 467, instead of at distal portion 506 or distal portion 511. In this case, the cavitation bubble tube 521 or outer tube 484 could be omitted such that the lumen 211 of the balloon catheter 201 would act as cavitation bubble chambers 520 and 491. As shown in FIG. 26, adapter 530 includes a distal portion 531, and a proximal portion 529. Distal portion 531 that includes rapid exchange lumen for guidewire functionality that doesn't require the distal lumen of a medical device catheter. As shown in FIG. 26, electrodes 517, 518, and 519 are positioned at the proximal portion 529, just proximal to the attachment mechanism 467 and just distal to the tubular extension 471. In this configuration, the shockwave generating electrodes can be positioned in the location of the inflatable balloon 202 of an angioplasty balloon catheter parent module 201, instead of in the distal portion 531, distal to the balloon of an angioplasty balloon catheter parent module. The cavitation bubble chamber region, in this case the region of the lumen 211 of the balloon catheter 201 where the electrode set 517, 518, and 519 are positioned, can be filled with a cavitation solution similar to cavitation bubble chamber 520 described previously. Distal portion 531 can also include an opening 539 at the distal end to facilitate filling with a cavitation solution by allowing any entrapped air bubbles or vapor bubbles to escape.

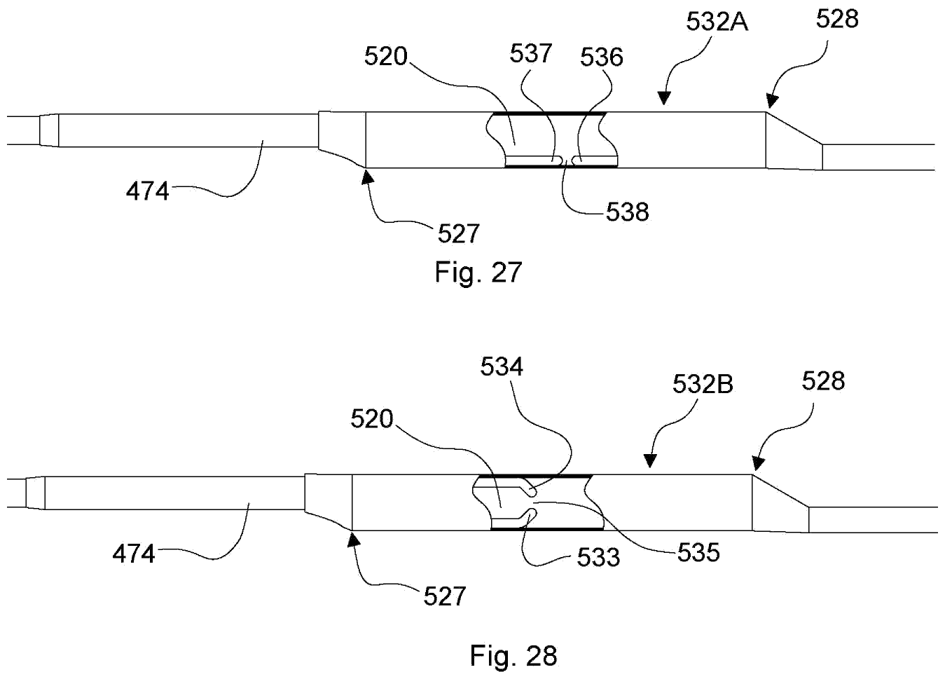

FIG. 27 is partial schematic view of an intravascular lithotripsy (IVL) adapter according to an aspect of the present disclosure, showing cutaway section view of distal portion 532A. Distal portion 532A includes a cavitation bubble chamber 520 with a distal end 528 and proximal end 527, runway 474, and co-linear, end-to-end electrodes, 536 and 537, within bubble cavitation chamber 520.

FIG. 28 is partial schematic view of an intravascular lithotripsy (IVL) adapter according to an aspect of the present disclosure, showing cutaway section view of distal portion 532B. Distal portion 532B includes a cavitation bubble chamber 520 with a distal end 528 and proximal end 527, runway 474, and parallel, end-to-end electrodes, 533 and 534, within bubble cavitation chamber 520. Instead of mostly parallel wire electrodes as shown in the example of adapters 505 and 510 of the present disclosure, the electrodes in the distal portions 506 and 511, respectively, can be configured in an end-to-end configuration of distal portion 532A and distal portion 532B as shown in FIG. 27 and FIG. 28. The parallel wire electrode configuration as shown in adapters 505 and 510 has advantages in ease of manufacturing but has drawbacks in that there is not a specific location where the arc or spark would occur along the length of the electrode, which may be problematic if the shock wave energy would need to be focused or precisely located. The end-to-end configuration as illustrated in FIG. 27 and FIG. 28 could be arranged to provide a more precise arc or spark location. FIG. 27 and FIG. 28 are longitudinal views with partial cutaway cross-sections of distal portions 532A and 532B to illustrate the interior of a cavitation bubble chamber 520 and alternate electrode configurations. FIG. 27, illustrates a pair of co-linear, end-to-end electrodes, 536 and 537, within bubble cavitation chamber 520. Applying a sufficiently high voltage potential difference between the set of electrodes 536 and 537 will induce arcing or sparking at the electrode gap 538 between the ends of electrodes 537 and 536 within the cavitation solution, and associated cavitation bubble. FIG. 28, illustrates a pair of parallel, end-to-end electrodes, 533 and 534, within bubble cavitation chamber 520. Applying a sufficiently high voltage potential difference between the set of electrodes 533 and 534 will induce arcing or sparking at the electrode gap 535 between the ends of electrodes 534 and 533 within the cavitation solution, and associated cavitation bubble.

Other suitable electrode set configurations include end-to-end and parallel electrode configurations, or electrode configurations that include combinations of end-to-end and parallel electrode. For example, an end of an electrode positioned or configured to create an electrode gap with a parallel electrode. The electrodes could be formed from wire, tubing, formed or cut conductive materials, sheet metal, or many other materials and forms. For example, where one or more of the electrodes could include one or more “teeth like” features, “pointy” features, sharpened features, laser cut features, shaped features, or screw thread type features, along the length or at the ends that can concentrate the current density for targeted or optimized electric arcing.

Calcium rich lesions within the vasculature is an issue affecting the cardiovascular health of many people. Lithotripsy, specifically the use of shockwaves to disrupt calcium can be an effective method to modify vascular calcium structures and improve outcomes during angioplasty procedures. According to the present disclosure, a novel modular catheter system and adapter are provided to enable lithotripsy procedures to be performed more effectively and flexibly by a physician.

While the invention has been described with reference to an exemplary embodiment(s), it will be understood by those skilled in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention is not limited to the particular embodiment(s) disclosed, but that the invention will include all embodiments falling within the scope of the appended claims.

1. An adapter for a medical device catheter, comprising:
a proximal portion and a distal portion;
at least two electrodes positioned to create an electrode gap between the electrodes;
an attachment mechanism configured to secure the adapter to a distal end of a medical device catheter; and
the proximal portion of the adapter comprising an electrical connector in electrical communication with the electrodes.

2. The adapter of claim 1, wherein the electrical connector extends proximally from a proximal end of the medical device catheter when the adapter is secured to the distal end of the medical device catheter.

3. The adapter of claim 1, wherein the distal portion of the adapter comprises the electrodes.

4. The adapter of claim 4, wherein the electrodes extend distally from a distal end of the medical device catheter when the adapter is secured to the medical device catheter.

5. The adapter of claim 4, wherein the distal portion further comprises a cavitation bubble chamber for containing a cavitation solution, and the electrodes are positioned within the cavitation bubble chamber to be in contact with the cavitation solution.

6. The adapter of claim 1, wherein the proximal portion of the adapter comprises the electrodes.

7. The adapter of claim 6, wherein the electrodes are positioned within a lumen of the medical device catheter when the adapter is secured to the distal end of the medical device catheter.

8. The adapter of claim 7, wherein the lumen is configured to contain a cavitation solution, and the electrodes are positioned within the lumen to be in contact with the cavitation solution.

9. The adapter of claim 1, wherein the medical device catheter is a balloon catheter.

10. The adapter of claim 1, wherein the electrodes are in a parallel configuration and the electrode gap is between parallel lengths of the electrodes.

11. The adapter of claim 10, wherein the electrodes comprise wire.

12. The adapter of claim 11, wherein the wire comprises insulated and uninsulated portions configured to create electrode gaps between the uninsulated portions.

13. The adapter of claim 1, wherein the electrodes are in an end-to-end configuration.

14. The adapter of claim 1, wherein the electrodes comprise a series of tubular electrode elements.

15. The adapter of claim 1, wherein the electrodes further comprise an intermediate electrode positioned intermediate to the at least two electrodes, thereby forming at least two electrode gaps.

16. The adapter of claim 1, further comprising an electrical conductor for facilitating the electrical communication between the electrical connector and the electrodes.

17. The adapter of claim 16, wherein a portion of the electrodes comprises the electrical conductor.

18. The adapter of claim 1, wherein the attachment mechanism comprises an interfacing element configured to secure the adapter to the distal end of the medical device catheter.

19. The adapter of claim 18, wherein the interfacing element comprises a compressible element configured to engage a lumen of the medical device catheter to secure the adapter to the distal end of the medical device catheter.

20. The adapter of claim 19, wherein the compressible element comprises a coil.
